(19) United States
(12) Patent Application Publication
(10) Pub. No.: US 2007/0148110 A1
(43) Pub. Date: Jun. 28, 2007

(54) Aqueous-alcoholic depigmenting gels comprising mequinol and adapalene

(75) Inventors: Leslie Zanutto, Paulhaguet (FR); Sandrine Orsoni, Mandelieu (FR); Laurent Fredon, Roquefort Les Pins (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 11/636,432

(22) Filed: Dec. 11, 2006

Related U.S. Application Data
(63) Continuation of application No. PCT/FR05/01393, filed on Jun. 7, 2005.

(30) Foreign Application Priority Data
Jun. 11, 2004 (FR) 0406338

(51) Int. Cl. A61K 8/35

(52) U.S. Cl. 424/62

(57) ABSTRACT

Stable, topically applicable cosmetic/pharmaceutical skin depigmentation compositions contain a combination depigmentation effective amount of mequinol and adapalene, and optionally, at least one sunscreen, formulated as aqueous-alcoholic gels or gel-creams in topically applicable, physiologically acceptable media therefor.

### CROSS-REFERENCE TO PRIORITY/PCT APPLICATIONS

This application claims priority under 35 U.S.C. § 119 of FR 04/06338, filed Jun. 11, 2004, and is a continuation of PCT/FR 2005/001393, filed Jun. 7, 2005 and designating the United States (published in the French language on Jan. 12, 2006 as WO 2006/003299 A1; the title and abstract were also published in English), each hereby expressly incorporated by reference and each assigned to the assignee hereof.

### BACKGROUND OF THE INVENTION

1. Technical Field of the Invention

The present invention relates to depigmenting compositions for cosmetic or pharmaceutical application comprising, formulated into a physiologically acceptable medium, mequinol (4-hydroxyanisole) and adapalene (6-[3-(1-adamantyl)-4-methoxyphenyl]-2-naphthoic acid), notably in dispersed form, also in the form of an aqueous-alcoholic gel or gel-cream.

2. Description of Background and/or Related and/or Prior Art

Among the therapeutic bioactive agents recommended for the treatment of cutaneous hyperpigmentation, phenolic derivatives such as mequinol and derivatives thereof have for decades been among the most effective actives.

However, phenolic derivatives are known to be sensitive to oxidation and to heat, such that formulations thereof quickly turn brown, and sometimes phase separation may even occur.

Moreover, as adapalene has poor solubility in water, it has to be dispersed in the composition of the formulation and therefore possible sedimentation of this active product is the main problem encountered when it must be included in a formulation. Thus, the difficulty is to obtain a formulation that is at the same time sufficiently fluid, yet has some viscosity in order to maintain the product in suspension and not flow, and containing adapalene in suspension.

In the present invention, adapalene has been successfully suspended owing to the aqueous-alcoholic gel or gel-cream form and the use of carbomer gels and surface-active wetting agents to overcome the problems of sedimentation.

In the prior art, sulfite salts are conventionally used for reducing the problem of formulations turning brown. However, they can alter the viscosity of formulations that are sensitive to electrolytes.

Notably, sulfite salts are known to break carbomer gels, leading to a drop in the viscosity-increasing power of the gelling agents and thus resulting in sedimentation of the actives.

### SUMMARY OF THE INVENTION

Novel topical pharmaceutical compositions containing mequinol and adapalene have now been developed, formulated as to be stable physically (without phase separation and without a significant drop in viscosity) and chemically (without altering the stability of the actives) and with optimized penetration of adapalene and mequinol into the skin.

Thus, it has now surprisingly been demonstrated that a formulation in the form of an aqueous-alcoholic gel or gel-cream containing excipients as described herein, provides good results with respect to physical and chemical stability of the active compounds. It also offers an excellent compromise from stability, notably resistance to temperature and to oxidation, efficacy, safety and cosmetic qualities.

Indeed, because of its composition and notably the presence of 2% to 10% of alcohol, the aqueous-alcoholic gel or gel-cream ensures that the composition and its components are stable, as well as being safe.

### BRIEF DESCRIPTION OF THE DRAWINGS

FIG. 1 is a graph showing the kinetics of mouse depigmentation scores as a function of treatment time for two formulations, including those according to the present invention, and

FIG. 2 is a bar graph showing the comparative depigmentation scores of the two formulations.

### DETAILED DESCRIPTION OF BEST MODE AND SPECIFIC/PREFERRED EMBODIMENTS OF THE INVENTION

Moreover, monitoring of the stability of the formulations presented in the examples given below shows that by combining the active compounds with sulfite salts and notably sodium metabisulfite and sodium sulfite, EDTA and alcohol (ethanol) reduces the browning of mequinol considerably. Without the sulfites, browning is observed at 55° C. after 1 month of storage, and in the absence of all of the compounds indicated above, browning occurs in just a few days at 55° C.

The solution to the problem of the drop in viscosity-increasing power of the carbomers, affected by sulfite salts, for suspension of adapalene, entails adding other gelling agents to the formulations.

The gelling agent or agents selected, alone or in combination, must have the following properties:
gelling of an aqueous phase, so as to form an aqueous gel with sufficient stiffness such that the final product does not flow when the container is inverted;
to provide sufficient viscosity to maintain the adapalene in suspension;
to have low sensitivity to electrolytes, i.e., not lose their gelling properties in the presence of electrolytes;
not break down over time, or at various storage temperatures (4° C.—room temperature (RT)—40° C.).

The formulations set forth in the following examples show that adapalene disperses easily and the dispersion remains homogeneous over time, aided by the network formed by the gelling agents and by addition of a surface-active wetting agent.

Notably, in particular the stability of the active phase of the compositions according to the invention highlights the efficacy of the product.

It has thus also been demonstrated that the depigmenting action of mequinol is increased synergistically by the presence of adapalene in the composition. Thus, the results presented in the examples demonstrate that the combination provides, advantageously, a quicker and more effective depigmenting effect.

A method of manufacture of the compositions according to the invention is also provided hereby.

The present invention therefore features depigmenting compositions comprising, formulated into a physiologically acceptable medium, mequinol and adapalene, in the form of an aqueous-alcoholic gel or gel-cream.

“Physiologically acceptable medium” means a medium that is compatible with the skin, the mucosae and/or the skin appendages.

“Depigmenting composition” means any composition comprising at least one active agent having skin depigmenting activity. This activity makes it possible to reduce the existing pigmentation of the skin.

“Aqueous-alcoholic gel” means an aqueous gel containing alcohol, water and at least one gelling agent.

“Aqueous-alcoholic gel-cream” means an aqueous gel containing an aqueous phase, a small proportion (from 0 to 20% to preferably 10%) of oil phase, and alcohol, said aqueous phase containing a gelling agent that is able to form a network that traps the oil droplets and keeps them in suspension.

The aqueous-alcoholic gel-cream is a formulation which combines the advantages of a gel (ease of application, quick release of the active agent, freshness on application) with those of a cream (comfortable for the skin on account of the small proportion of oil phase, no dryness of the skin).

The compositions according to the invention preferably contain from 2% to 10% of alcohol and more preferably 5%.

Among the alcohols, non-limiting examples thereof are ethanol, isopropanol, and butanol. Ethanol is particularly preferred.

Advantageously, the compositions according to the invention also contain a chelating agent, a surface-active wetting agent and one or more gelling agents.

The compositions according to the invention also advantageously contain one or more of the following ingredients:
a) a carbomer;
b) one or more other gelling agents;
c) an antioxidant;
d) an oil phase;
e) a moisturizer/emollient;
f) an anti-irritant;
g) a pH neutralizer;
h) a preservative.

Preferably, the aqueous-alcoholic gels or gel-creams according to the present invention comprise a carbomer and one or more other gelling agents or said carbomer and one or more other carbomers. Indeed, as already indicated, these compounds provide the composition with suitable viscosity, while maintaining the adapalene in suspension.

Among the carbomers and other possible gelling agents, the following are representative non-limiting examples: carbomer 1382 marketed under the trademark Carbopol 1382 by BF Goodrich or the acrylate/C 10-C30 alkyl acrylate crosspolymer, marketed under the trademark Pemulen TR1 by BF Goodrich, xanthan gum such as Keltrol T marketed by Kelco, carbopol 980, carbopol 981, carbopol Ultrez 10, carbopol EDT 2020, carbopol 974, hydroxypropylcellulose such as the product marketed under the trademark Natrosol HHX 250 by Aqualon, and the acrylamide/sodium acryloyldimethyltaurate copolymer and isohexadecane and polysorbate 80 marketed under the trademark Simulgel 600 by Seppic.

Among the gelling agents, preferred is the combination of the carbomer/acrylate/C10-C30 alkyl acrylate crosspolymer with xanthan gum and hydroxyethylcellulose or, alternatively, the combination of carbomer 1382 with xanthan gum and carbomer 981.

Among the antioxidants, the following are representative non-limiting examples: ascorbic acid and its salts, tocopherols and sulfite salts such as sodium metabisulfite, sodium sulfite.

The oil phase of the composition according to the invention can comprise, for example, vegetable, mineral, animal or synthetic oils, silicone oils, and mixtures thereof.

Examples of mineral oils are the paraffin oils of various viscosities such as Primol 352, Marcol 82, Marcol 152 marketed by Esso.

The following are representative vegetable oils: sweet almond oil, palm oil, soya oil, sesame oil, sunflower oil.

The following are representative animal oils: lanolin, squalene, fish oil, mink oil.

As synthetic oils, representative are esters such as cetearyl isononanoate such as the product marketed under the trademark Cetiol SN by Cognis France, diisopropyl adipate such as the product marketed under the trademark Ceraphyl 230 by ISF, isopropyl palmitate such as the product marketed under the trademark Crodamol IPP by Croda, caprylic capric triglyceride such as Miglyol 812 marketed by Huls/Lambert Rivière.

As silicone oils, representative are a dimethicone such as the product marketed under the trademark Dow Corning 200 fluid, a cyclomethicone such as the product marketed under the trademark Dow Corning 244 fluid by Dow Corning or the product marketed under the trademark Mirasil CM5 by SACI-CFPA.

It will also be possible to use solid fats, such as natural or synthetic waxes. In this case one skilled in the art will adapt the temperature of heating of the preparation in relation to the presence or absence of these solids.

For the depigmenting compositions of the aqueous-alcoholic gel-cream type according to the invention, paraffin oils and more particularly Marcol 152 are preferred.

For better dispersion of adapalene, the compositions according to the invention advantageously contain one or more surface-active wetting agents at concentrations from 0.01% to 10% to more preferably from 0.1% to 5%.

Preferably, they are surfactants having an HLB (Hydrophilic Lipophilic Balance) from 7 to 9, or, alternatively, non-ionic surfactants such as polyoxyethylene and/or polyoxypropylene copolymers.

The following are representative non-limiting examples of surface-active wetting agents: compounds of the poloxamer class and more particularly Poloxamer 124 and Poloxamer 182.

The surface-active wetting agent particularly preferred is Poloxamer 124.

The following are representative examples of chelating agents: ethylenediamine tetraacetic acid (EDTA), calcium disodium edetate, sodium edetate, disodium edetate and preferably disodium edetate and EDTA.

The compositions can additionally contain additives that are usually employed in the cosmetic or pharmaceutical field, such as a neutralizing agent, a moisturizer and/or co-solvent, an emollient, a soothing agent, a preservative, a pH corrector, or mixtures thereof.

Of course, one skilled in this art will select this or these optional additional compounds, and/or their amount, in such manner that the advantageous properties of the compositions according to the invention are not, or substantially not, adversely affected.

These additives can be present in the compositions in a proportion from 0.001% to 20 wt. % relative to the total weight of the composition.

The following are representative examples of moisturizers/emollients: glycerol, sorbitol, propylene glycol.

An exemplary co-solvent is macrogol 400.

Anti-irritant and/or “soothing” agents can also be added to the formulations, such as strontium nitrate, shea butter, potassium salt of 18-beta-glycyrrhetinic acid, acid dipotassium glycyrrhizate, tea tree oil, enoxolone, alpha-tocopherol acetate, allantoin, talc.

The following are representative examples of pH neutralizing agents for obtaining a suitable pH: an amine base such as triethanolamine, diethanolamine, tromethamine, tromethamol or many other bases such as sodium hydroxide.

Exemplary preservatives include benzalkonium chloride, phenoxyethanol, benzyl alcohol, diazolidinyl urea, parabens, or mixtures thereof.

However, the preferred compositions according to the invention advantageously do not contain a preservative.

The active agents according to the invention are mequinol (4-hydroxyanisole) as well as its precursors and/or derivatives thereof and adapalene (6-[3-(1-adamantyl)-4-methoxyphenyl]-2-naphthoic acid), notably in dispersed form, as well as its precursors and/or derivatives thereof to which it is possible to add other agents as was explained previously. By dispersed form is meant distribution of a solid of variable granulometry in a liquid medium.

Of course, the amount of the active agents in the compositions according to the invention will depend on the combination selected and therefore, in particular, on the quality of the desired treatment. Preferably, the amount of adapalene is from 0.0001% and 20%, more preferably from 0.001% and 10%.

The compositions of the aqueous-alcoholic gel or gel-cream type according to the invention offer good cutaneous tolerance. Advantageously, they can be spread more easily than a viscous emulsion and they leave a pleasant sensation of freshness.

More particularly, this invention features aqueous-alcoholic depigmenting gels or gel-creams comprising one or more of the following ingredients:
from 0.01% to 5% of mequinol;
from 0.10% to 2% of adapalene;
from 2% to 10% of ethanol;
from 0.01% to 2% of one or more gelling agents;
from 0% to 1% of an antioxidant;
from 0.01% to 20% of chelating agent;
from 0% to 20% of an oil phase.

Preferred compositions according to the invention comprise:
2% of mequinol;
0. 10% to 2% of adapalene;
5% of ethanol;
from 1% to 2% of one or more gelling agents;
from 0.1% to 0.5% of an antioxidant;
0.10% of EDTA;
from 0% to 15% of an oil phase.

Particularly preferred compositions according to the invention comprise:
2% of mequinol;
0.10% of adapalene;
5% of ethanol;
1% to 2% of one or more gelling agents;
0.1% to 0.4% of an antioxidant and more preferably of sulfite salts;
0.10% of EDTA;
from 5% to 15% of an oil phase.

The present invention also features compositions as defined above and containing a chemical or physical sun filter.

“Sun filter” means a chemical or physical sun filter/sunscreen and mixtures thereof, and the following are representative non-limiting examples: physical sun filters such as titanium dioxide, zinc oxide and chemical sun filters such as octocrylene, ethylhexyl methoxycinnamate, octyl salicylate, avobenzone, oxybenzone, ecamsule, drometrizole trisiloxane.

Each sun filter can be added at a concentration in the range from 0.001% to 20 wt. % relative to the total weight of the composition and preferably from 0.001% and 5%.

The present invention also features the composition as described above as medicinal products.

This invention also features a method of preparation of a composition of the aqueous-alcoholic gel or gel-cream type, comprising the following stages in succession:
a) prepare a formulation phase with water and stir in a Rayneri stirrer, then pour in the chelating agent and stir until dissolved;
b) heat the mixture from stage a) to 60° C. and sprinkle in the gelling agent or agents, stirring until homogeneous;
c) leave the mixture to return to room temperature, while stirring in a Rayneri stirrer;
d) prepare, in a separate beaker, a first active phase comprising mequinol and alcohol, with magnetic stirring until completely dissolved;
e) add this first active phase to the formulation phase after it has returned to room temperature, and maintain stirring;
f) prepare, in a separate beaker, a second active phase comprising adapalene, surface-active wetting agent and moisturizer, stirring until a smooth, homogeneous dispersion is obtained;
g) then add this second active phase to the formulation phase after it has returned to room temperature, and maintain stirring;
h) neutralize with a neutralizing agent to obtain the desired pH while stirring in a Rayneri stirrer;
i) add the antioxidants to the formulation phase, while stirring.

The native pH of the mixture is verified and is corrected if necessary with a solution of a neutralizing agent.

Any optional additives can be incorporated in relation to their chemical nature during one of the stages of the method of preparation described above.

According to a particular embodiment of the method of the invention, a moisturizer and/or an anti-irritant can optionally be added in stage a) at the same time as the chelating agent.

In another particular embodiment of the method of the invention, an oil phase obtained by mixing an oil, a surfactant and a preservative heated on a water bath to 60° C. is added to the formulation phase obtained at the end of stage b).

Depending on the physicochemical characteristics of the sun filter/sunscreen, one skilled in this art will take care to incorporate it during one of the stages defined above.

“Formulation phase” means the mixture of a group of ingredients introduced together in a single phase.

“Active phase” means a formulation phase containing one or more actives.

The present invention also features the use of the novel compositions as described previously in cosmetics and in dermatology.

In particular, the invention relates to the use of a composition as described previously for the manufacture of a pharmaceutical preparation intended for the treatment and/or prevention of dermatologic conditions associated with disorders of pigmentation, whether regime or regimen.

The compositions of the invention are particularly suitable for the treatment and/or prevention of dermatologic conditions associated with disorders of pigmentation such as melasma, chloasma, lentigines, lentigo senile, vitiligo, freckles, post-inflammatory hyperpigmentations due to abrasion, burns, scars, a dermatosis, a contact allergy, naevi, genetically-determined hyperpigmentations, hyperpigmentations of metabolic or medication-induced origin, melanomas or all other hyperpigmentation lesions.

The compositions according to the invention also find application in the cosmetic field, in particular for preventing and/or combating the harmful effects of the sun and/or photo-induced or chronological aging of the skin and its appendages.

The compositions according to the invention also find application in body and hair hygiene.

The present invention also features a regime or regimen of non-therapeutic cosmetic treatment for embellishment of the skin and/or improvement of its surface appearance, wherein an aqueous-alcoholic gel or gel-cream according to the invention comprising mequinol and adapalene, and optionally a sun filter, is topically applied onto the skin and/or its appendages.

In order to further illustrate the present invention and the advantages thereof, the following specific examples are given, including those illustrating the stability of the subject compositions, it being understood that same are intended only as illustrative and in nowise limitative. In said examples to follow, all parts and percentages are given by weight, unless otherwise indicated.

### EXAMPLES

In the following compositions (Examples 1, 3, 5, 7, 9 and 11), the proportions of the various constituents are expressed as percentage by weight relative to the total weight of the composition.

### Example 1

### Gel Formulation

| Purified water | Qsf 100% |
|---|---|
| EDTA | 0.10 |
| Glycerol | 5.00 |
| Xanthan Gum | 0.40 |
| Acrylate/C10 C30 Alkyl Acrylate | 0.60 |
| Crosspolymer | |
| Ethanol | 5.00 |
| Mequinol | 2.00 |
| Phenoxyethanol | 1.00 |
| Propylene glycol | 5.00 |
| Poloxamer 124 | 0.20 |
| Adapalene | 0.10 |
| Triethanolamine | 1.30 |
| Purified water | 5.00 |
| Sodium metabisulfite | 0.20 |
| Sodium sulfite | 0.20 |

The gel formulation is prepared by the following method:
a) formulation phase:
   Put most of the water in the main beaker and stir in a Rayneri stirrer. Add the chelating agent, the anti-irritant and glycerol, then continue stirring until dissolved.
   Heat to 60° C. to facilitate dispersion of the gelling agents.
   Sprinkle the gelling agent or agents in the formulation phase and continue stirring until homogeneous.
   Then leave to return to room temperature (RT), stirring continuously.
b) active phase:
   weigh the depigmenting agent and the ethanol in a separate beaker, then stir with a magnetic stirrer until completely dissolved;
   finally add to the formulation phase which has returned to room temperature (RT), while stirring in a Rayneri stirrer;
   weigh the adapalene, propylene glycol and poloxamer 124 in a separate beaker, then stir in an Ultra-turax at 20500 rev/min for 15 min until a smooth, homogeneous dispersion is obtained;
   finally add to the formulation phase that has returned to RT, while stirring in a Rayneri stirrer.
c) Neutralize with a base so as to obtain a pH of 6+/−0.3 while stirring in a Rayneri stirrer.
d) Finally add the antioxidants to the formulation phase while stirring in a Rayneri stirrer.

### Example 2

### Physical and Chemical Stability of the Gel Formulation According to Example 1

The physical stability of the gel formulation according to Example 1 is measured for 3 months at room temperature (RT), at 4° C., at 40° C. and at 55° C.:

Characteristics of the formulation at T0:

| | | | | | | |
|---|---|---|---|---|---|---|
| pH | 5.93 | Macroscopic | White, fluid gel | Centrifugation | 3000 rev/min | compliant |
| | | appearance | | | | |
| | | Microscopic | Homogeneous | | 10000 rev/min | compliant |
| | | appearance | Dispersion of | | | |
| | | | Adapalene | | | |

| | | T1 months | T2 months | T3 months |
|---|---|---|---|---|
| RT | pH | 5.69 | 5.41 | 5.35 |
| | centrifugation | Compliant | Compliant | Compliant |
| | Macroscopic | Compliant | Compliant | Compliant |
| | appearance | | | |
| 4° C. | Macroscopic | Compliant | Compliant | Compliant |
| | appearance | | | |
| | Microscopic | Compliant | Compliant | Compliant |
| | appearance | | | |
| 40° C. | Macroscopic | Compliant | Compliant | Compliant |
| | appearance | | | |
| | Microscopic | Compliant | Compliant | Compliant |
| | appearance | | | |
| 55° C. | Macroscopic | Compliant, more | Compliant, more | Compliant, |
| | appearance | fluid than at 4° C. | fluid than at 4° C. | more fluid than |
| | | | | at 4° C. |

“Compliant” in the above means that the characteristics of the composition measured at 1, 2 or 3 months comply with those obtained at T0.

The chemical stability of the gel formulation according to Example 1 is measured by HPLC over 3 months at RT and at 40° C.:

| | | RT | 40° C. |
|---|---|---|---|
| CHEMICAL | T0 | Mequinol: 94.4% | Mequinol: / |
| STABILITY | | Adapalene: 92.6% | Adapalene: / |
| | T1 M | Mequinol: 96.6% | Mequinol: 93.9% |
| | | Adapalene: 91.7% | Adapalene: 92.6% |
| | T2 M | Mequinol: 93.6% | Mequinol: 93.8% |
| | | Adapalene: 93.0% | Adapalene: 93.% |
| | T3 M | Mequinol: 93.9% | Mequinol: 93.5% |
| | | Adapalene: 92.8% | Adapalene: 94.0% |

The results show that this composition is stable physically and chemically for 3 months and at all temperatures.

Furthermore, no browning of the formula at 40° C. is observed after 3 months.

### Example 3

### Gel-cream Formulation

| Purified water | Qsf 100% |
|---|---|
| EDTA | 0.10 |
| Glycerol | 5.00 |
| Xanthan gum | 0.40 |
| Acrylate/C10 C30 Alkyl Acrylate Crosspolymer | 0.60 |
| Mineral oil | 10.00 |
| Ceteareth 20 | 0.50 |
| Phenoxyethanol | 1.00 |
| Ethanol | 5.00 |
| Mequinol | 2.00 |
| Propylene glycol | 5.00 |
| Poloxamer 124 | 0.20 |
| Adapalene | 0.20 |
| Triethanolamine | 0.40 |
| Purified water | 5.00 |
| Sodium metabisulfite | 0.20 |
| Sodium sulfite | 0.20 |

The gel-cream formulation is prepared by the following method:
a) aqueous formulation phase:
   Put most of the water in the main beaker and stir in a Rayneri stirrer.
   Add the chelating agent, the anti-irritant and glycerol, then continue stirring until dissolved.
   Heat to 60° C. to facilitate dispersion of the gelling agents.
   Sprinkle in the gelling agent or agents and continue stirring until homogeneous.
b) Oil phase:
   Weigh the mineral oil, the surfactant and the preservative in a separate beaker.
   Heat on a water bath to 60° C.
   Then add to aqueous phase a) while stirring sufficiently in a Rayneri stirrer.
   Leave the emulsion to return to room temperature.
c) Active phase
   weigh the mequinol and the ethanol in a separate beaker, then stir with a magnetic stirrer until completely dissolved;
   finally add to the formulation phase which has returned to room temperature (RT), while stirring in a Rayneri stirrer;
   weigh the adapalene, propylene glycol and poloxamer 124 in a separate beaker, then stir in an Ultra-turax at 20500 rev/min for 15 min until a smooth, homogeneous dispersion is obtained;
   finally add to the formulation phase that has returned to RT, while stirring in a Rayneri stirrer.
d) Neutralize with a base to obtain the desired pH while stirring in a Rayneri stirrer.
e) Finally add the solution of sulfites to the formulation phase while stirring in a Rayneri stirrer.

### Example 4

### Physical and Chemical Stability of the Gel-cream Formulation According to Example 3

The chemical stability of the gel-cream formulation according to Example 3 is measured by HPLC for 3 months at room temperature (RT) and at 40° C.:

CHARACTERISTICS OF THE FORMULATION AT T0

| | | | | | | | | |
|---|---|---|---|---|---|---|---|---|
| pH | 5.80 | Tau θ | 13 | Macroscopic | Shiny, white | Centrifugation | 3000 rev/min | Exudate |
| | | | | appearance | gel-cream | | | |
| | | | | Microscopic | Homogeneous | | | |
| | | | | appearance | dispersion of | | 10000 rev/min | Exudate |
| | | | | | adapalene | | | |

The yield point (tau θ) is the force required (minimum shear stress) to overcome the forces of cohesion of the van der Waals type and cause flow. The yield point is compared with the value found at 4 s-1.

| | | RT | 40° C. |
|---|---|---|---|
| CHEMICAL | T0 | Mequinol: 100.5% | Mequinol: / |
| STABILITY | | Adapalene: 99.2% | Adapalene: / |
| | T1 M | Mequinol: 99.8% | Mequinol: 98.3% |
| | | Adapalene: 99.8% | Adapalene: 99.1% |
| | T2 M | Mequinol: 99.7% | Mequinol: 99.4% |
| | | Adapalene: 101.0% | Adapalene: 100.3% |
| | T3 M | Mequinol: 100.0% | Mequinol: 99.6% |
| | | Adapalene: 100.0% | Adapalene: 100.5% |

This composition is chemically stable for 3 months at all the temperatures.

### Example 5

### Other Gel-cream Formulation

| Purified water | Qsf 100% |
|---|---|
| EDTA | 0.10 |
| Allantoin | 0.20 |
| Glycerol | 5.00 |
| Xanthan gum | 0.40 |
| Hydroxyethylcellulose | 0.70 |
| Carbomer 1382 | 0.20 |
| Mineral oil | 10.00 |
| Sorbitan monooleate | 1.00 |
| Ethanol | 5.00 |
| Mequinol | 2.00 |
| Propylene glycol | 5.00 |
| Poloxamer 124 | 0.20 |
| Adapalene | 0.20 |
| Solution of tris amino at 10% | 1.30 |
| Purified water | 5.00 |
| Sodium metabisulfite | 0.10 |
| Sodium sulfite | 0.10 |

This formulation is prepared according to the method described in Example 3.

### Example 6

### Physical and Chemical Stability of the Gel-cream Formulation According to Example 5

The physical stability of the gel-cream formulation according to Example 5 is measured for 3 months at room temperature (RT), at 4° C. and at 40° C.:

CHARACTERISTICS OF THE FORMULATION AT T0

| | | | | | | | | |
|---|---|---|---|---|---|---|---|---|
| pH | 5.97 | Tau θ | 24 | Macroscopic | Shiny, oily, | Centrifugation | 3000 rev/min | SMOOTH |
| | | | | appearance | white gel- | | | |
| | | | | | cream | | | |
| | | | | Microscopic | Fine emulsion, | | 10000 rev/min | SMOOTH |
| | | | | appearance | droplets from | | | |
| | | | | | 2.5μ to 7.5μ. | | | |
| | | | | | Homogeneous | | | |
| | | | | | dispersion of | | | |
| | | | | | the adapalene | | | |

| | | T1 months | T2 months | T3 months |
|---|---|---|---|---|
| RT | pH | 5.57 | 5.39 | 5.27 |
| | Centrifugation | Compliant | Compliant | Compliant |
| | Macroscopic | Compliant | Compliant | Compliant |
| | appearance | | | |
| | Microscopic | Compliant. | Compliant. | Compliant. |
| | appearance | Droplets from | Droplets from | Droplets from |
| | | 2.5μ to 12.5μ. | 2.5μ to 10μ. | 2.5μ to 7.5μ. |
| | ⊖₀ (tau) | 45 | 25 | 20 |
| 4° C. | Macroscopic | Compliant | Compliant | Compliant |
| | appearance | | | |
| | Microscopic | Compliant. | Compliant. | Compliant. |
| | appearance | Droplets from | Droplets from | Droplets from |
| | | 2.5μ to 6μ. | 2.5μ to 15μ. | 2.5μ to 10μ. |
| 40° C. | pH | 6.05 | 5.96 | 5.66 |
| | Macroscopic | Compliant | Compliant | Compliant |
| | appearance | | | |
| | Microscopic | Compliant. | Compliant. | Compliant. |
| | appearance | Droplets from | Droplets from | Droplets from |
| | | 2.5μ to 12.5μ. | 2.5μ to 8μ. | 2.5μ to 7.5μ. |

The chemical stability of the gel-cream formulation according to Example 5 is measured by HPLC for 2 months at room temperature (RT) and at 40° C.:

| | | RT | 40° C. |
|---|---|---|---|
| CHEMICAL | T0 | Mequinol: 99.8% | Mequinol: / |
| STABILITY | | Adapalene: 100.9% | Adapalene: / |
| | T1 M | Mequinol: 100.2% | Mequinol: 107.0% |
| | | Adapalene: 101.1% | Adapalene: 104.0% |
| | T2 M | Mequinol: 102.9% | Mequinol: 101.0% |
| | | Adapalene: 103.5% | Adapalene: 103.1% |

This composition is stable physically and chemically at all the temperatures.

### Example 7

### Other Gel-cream Formulation

| Purified water | Qsf 100% |
|---|---|
| EDTA | 0.10 |
| Allantoin | 0.20 |
| Glycerol | 5.00 |
| Xanthan gum | 0.40 |
| Hydroxyethylcellulose | 0.70 |
| Acrylate/C10 C30 Alkyl Acrylate Crosspolymer | 0.20 |
| Mineral oil | 10.00 |
| Sorbitan monooleate | 1.00 |
| Ethanol | 5.00 |
| Mequinol | 2.00 |
| Propylene glycol | 5.00 |
| Poloxamer 124 | 0.20 |
| Adapalene | 0.20 |
| Solution of tris amino at 10% | 1.30 |
| Purified water | 5.00 |
| Sodium metabisulfite | 0.05 |
| Sodium sulfite | 0.05 |

This formulation is prepared according to the method described in Example 3.

### Example 8

### Physical and Chemical Stability of the Gel-cream Formulation According to Example 7

The physical stability of the gel-cream formulation according to Example 7 is measured for 2 months at room temperature (RT), at 4° C. and at 40° C.:

CHARACTERISTICS OF THE FORMULATION AT T0

| | | | | | | | | |
|---|---|---|---|---|---|---|---|---|
| pH | 6.16 | Tau θ | 65 | Macroscopic | Shiny, oily, | Centrifugation | 3000 rev/min | SMOOTH |
| | | | | appearance | white gel- | | | |
| | | | | | cream | | | |
| | | | | Microscopic | Fine emulsion, | | 10000 rev/min | SMOOTH |
| | | | | appearance | droplets from | | | |
| | | | | | 2.5μ to 7.5μ. | | | |
| | | | | | Homogeneous | | | |
| | | | | | dispersion of | | | |
| | | | | | the adapalene | | | |

| | | T1 months | T2 months | T3 months |
|---|---|---|---|---|
| RT | pH | 5.95 | 5.92 | 5.67 |
| | centrifugation | SMOOTH | SMOOTH | SMOOTH |
| | Macroscopic | Compliant | Compliant | Compliant |
| | appearance | | | |
| | Microscopic | Compliant | Compliant | Compliant |
| | appearance | Droplets | Droplets | Droplets |
| | | 2.5μ to 5μ. | 2.5μ to 12.5μ. | 2.5μ to |
| | | | | 12.5μ. |
| | θ₀ (tau) | 52 | 41 | 54 |
| 4° C. | Macroscopic | Compliant | Compliant | Compliant |
| | appearance | | | |
| | Microscopic | Compliant | Compliant | Compliant |
| | appearance | Droplets | Droplets | Droplets |
| | | 2.5μ to | 2.5μ to | 2.5μ |
| | | 10μ. | 12.5μ. | |
| 40° C. | pH | 5.85 | 5.69 | 5.30 |
| | Macroscopic | Compliant | Compliant | Compliant |
| | appearance | | | |
| | Microscopic | Compliant | Compliant | Compliant |
| | appearance | Droplets | Droplets | Droplets |
| | | 2.5μ to 5μ. | 2.5μ to7.5μ. | 2.5μ to |
| | | | | 7.5μ. |

The chemical stability of the gel-cream formulation according to Example 7 is measured by HPLC for 1 month at room temperature (RT) and at 40° C.:

| | | RT | 40° C. |
|---|---|---|---|
| CHEMICAL | T0 | Mequinol: | Mequinol: / |
| STABILITY | | 100.6% | Adapalene: / |
| | | Adapalene: | |
| | | 98.0% | |
| | T1 M | Mequinol: | Mequinol: |
| | | 100.7% | 106.2% |
| | | Adapalene: | Adapalene: |
| | | 98.4% | 103.4% |

This composition is stable physically and chemically at all the temperatures.

### Example 9

### Other Gel-cream Formulation

| | |
|---|---|
| Purified water | Qsf 100% |
| Methyl paraben | 0.20 |
| Disodium edetate | 0.10 |
| Glycerol | 5.00 |
| Allantoin | 0.20 |
| Carbomer 1382 | 0.60 |
| Carbomer (Carbopol 981NF) | 0.20 |
| Xanthan gum | 0.40 |
| Mequinol | 2.00 |
| Butyl hydroxytoluene | 0.10 |
| Ethanol 95% | 5.00 |
| Liquid paraffin | 10.00 |
| Sorbitan monooleate | 1.00 |
| Poloxamer 124 | 0.20 |
| Propylene glycol | 5.00 |
| Adapalene | 0.10 |
| Sodium sulfite | 0.05 |
| Sodium bisulfite | 0.05 |
| Triethanolamine | qs pH 4.5 |

This formulation is prepared according to the method described in Example 3.

### Example 10

### Stability of the Gel-cream Formulation According to Example 9

The physical stability of the gel-cream formulation according to Example 9 is measured for 3 months at room temperature (RT), at 45° C. and at 55° C.:

CHARACTERISTICS OF THE FORMULATION AT T0

| | | | |
|---|---|---|---|
| pH | 4.56 | Macroscopic | Shiny, white gel-cream |
| | | appearance | |
| | | Microscopic | Fine emulsion, droplets from 2.5μ to |
| | | appearance | 15μ. Homogeneous distribution of the |
| | | | adapalene |

| | | T1 months | T2 months | T3 months |
|---|---|---|---|---|
| RT | pH | 4.45 | 4.56 | 4.60 |
| | Macroscopic | Compliant | Compliant | Compliant |
| | appearance | | | |
| | Microscopic | Compliant | Compliant | Compliant |
| | appearance | | | |
| 45° C. | Macroscopic | Compliant | Compliant | Compliant |
| | appearance | | | |
| | Microscopic | Compliant | Compliant | Compliant |
| | appearance | | | |
| 55° C. | Macroscopic | Compliant | Compliant | Compliant |
| | appearance | | | |

This composition is physically stable (pH, viscosity) at all the temperatures for 3 months.

Moreover, no browning of the formula at 55° C. is observed after 3 months.

### Example 11

### Other Gel-cream Formulation with Sun Filter

| Purified water | Qsf 100% |
|---|---|
| EDTA | 0.10 |
| Glycerol | 5.00 |
| Ecamsule | 2.00 |
| Xanthan gum | 0.40 |
| Acrylate/C10 C30 Alkyl Acrylate Crosspolymer | 0.60 |
| Mineral oil | 5.00 |
| Octocrylene | 5.00 |
| Ceteareth 20 | 0.50 |
| Phenoxyethanol | 1.00 |
| Ethanol | 5.00 |
| Mequinol | 2.00 |
| Propylene glycol | 5.00 |
| Poloxamer 124 | 0.20 |
| Adapalene | 0.20 |
| Triethanolamine | 0.40 |
| Purified water | 5.00 |
| Sodium metabisulfite | 0.20 |
| Sodium sulfite | 0.20 |

The gel-cream formulation is prepared according to the method described in Example 3.

The sun filters are added during stage b).

### Example 12

### Measurement of the Depigmenting Activity of the Combination Adapalene and Mequinol in the SKH2 Mouse:

The purpose of the present study is to evaluate the depigmenting activity of a composition comprising either (i) 2% of mequinol, (ii) 0.1% of adapalene or (iii) the combination of both of them (composition according to the invention) on the skin of the tail of the SKH2 mouse after 4 weeks of topical application.

The two formulations gel and gel-cream are also compared. The two formulations (20 μl) are applied topically on the tails of SKH2 mice separated into two groups (female mice about 9 weeks old) at the rate of one application per day for 5 days over a period of 4 weeks.

Evaluation is based on various clinical observations: once a week, the pigmentation is evaluated as a score on a scale from 0 to 4. The basis of scoring is as follows:
0: natural pigmentation
   Depigmentation scale: scores −1 to −4
−1: slight depigmentation
−2: moderate depigmentation
−3: marked depigmentation
−4: total depigmentation

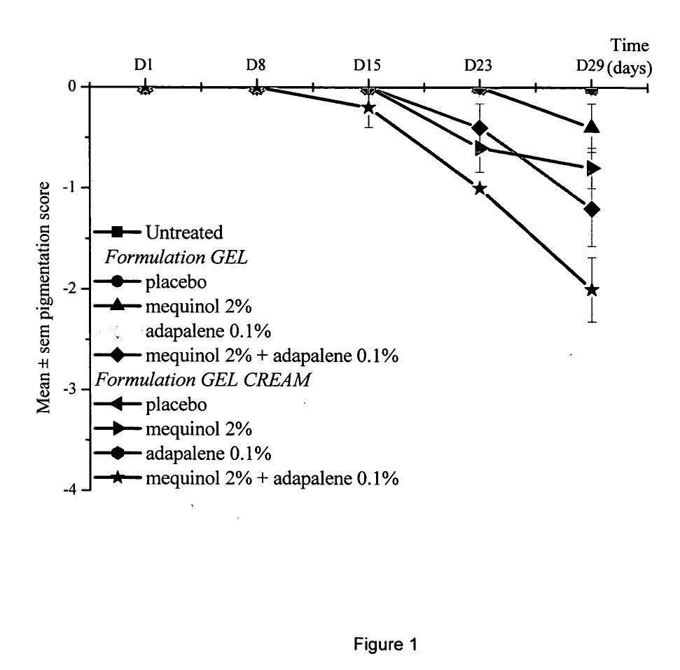

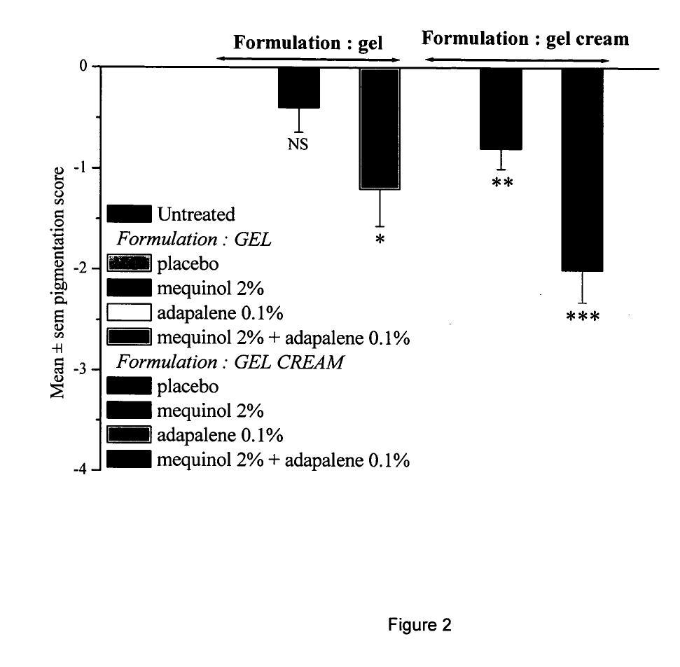

The results are shown in FIGS. 1 and 2.

FIG. 1 shows the kinetics of the mouse skin depigmentation scores as a function of the treatment time for the two formulations with:
(▪) skin not treated,
for the GEL formula: (●) skin treated with placebo, (▴) skin treated with Mequinol 2%, (▾) skin treated with Adapalene 0.1%, (⋄) skin treated with the combination Mequinol 2% +Adapalene 0.1%;
for the GEL-CREAM formula: ( [symbol] ) skin treated with placebo, ( [symbol] ) skin treated with Mequinol 2%, ( [symbol] ) skin treated with Adapalene 0.1%, (★) skin treated with the combination Mequinol 2%+Adapalene 0.1%.

FIG. 2 shows the comparative depigmentation scores of the two formulations with:
(▬) skin not treated
for the GEL formula: ( [symbol] ) skin treated with the combination Mequinol 2% +Adapalene 0.1%.
for the GEL-CREAM formula: ( [symbol] ) skin treated with Adapalene 0.1%, (▬) skin treated with the combination Mequinol 2+Adapalene 0.1%.

The results of the study show that after 4 weeks, the composition comprising 2% mequinol has a significant depigmenting effect, which is increased when 0.1% of adapalene is applied in combination.

Adapalene alone at 0.1% does not have a depigmenting effect, since the bar chart shows a score equal to 0 for the gel formulation and the gel-cream formulation. The same score equal to 0 is also recorded for the controls (untreated mice and mice treated with placebo).

The depigmenting effect is quicker and more pronounced with the gel-cream formulation and especially in the case of the combination of Mequinol with Adapalene.

The results show a synergistic effect on depigmenting activity from Mequinol and Adapalene. In particular, the combination of Mequinol 2% with Adapalene 0.1% has a quicker and more pronounced depigmenting effect than Mequinol alone.

The formulations according to Examples 1, 3, 5, 7 and 9 can be applied once or twice a day until there is total depigmentation, for the treatment of lentigines, chloasma or melasma.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A stable, topically applicable cosmetic/pharmaceutical skin depigmenting composition comprising a combination depigmentation effective amount of mequinol and adapalene, formulated as an aqueous-alcoholic gel or gel-cream in a topically applicable, physiologically acceptable medium therefor.

2. The aqueous-alcoholic gel or gel-cream as defined by claim 1, comprising from 2% to 10% by weight of at least one alcohol.

3. The aqueous-alcoholic gel or gel-cream as defined by claim 2, comprising ethanol, isopropanol and/or butanol.

4. The aqueous-alcoholic gel or gel-cream as defined by claim 1, further comprising at least one chelating agent, at least one surface-active wetting agent and at least one gelling agent.

5. The aqueous-alcoholic gel or gel-cream as defined by claim 1, further comprising at least one of the following ingredients:
a) a carbomer;
b) at least one other gelling agent;
c) an antioxidant;
d) an oil phase;
e) a moisturizer/emollient;
f) an anti-irritant;
g) a pH neutralizer;
h) a preservative.

6. The aqueous-alcoholic gel or gel-cream as defined by claim 5, comprising a carbomer and at least one other gelling agent.

7. The aqueous-alcoholic gel or gel-cream as defined by claim 5, comprising at least two different carbomers.

8. The aqueous-alcoholic gel or gel-cream as defined by claim 1, further comprising at least one sunscreen.

9. The aqueous-alcoholic gel or gel-cream as defined by claim 1, comprising:
from 0.01% to 5% of mequinol;
from 0.10% to 2% of adapalene;
from 2% to 10% of ethanol;
from 0.01% to 2% of at least one gelling agent;
from 0% to 1% of an antioxidant;
from 0.01% to 20% of a chelating agent;
from 0% to 20% of an oily liquid phase.

10. The aqueous-alcoholic gel or gel-cream as defined by claim 1, comprising:
2% of mequinol;
0.10% to 2% of adapalene;
5% of ethanol;
from 1% to 2% of at least one gelling agent;
from 0.1% to 0.5% of an antioxidant;
0.10% of EDTA;
from 0% to 15% of an oily liquid phase.

11. The aqueous-alcoholic gel or gel-cream as defined by claim 1, comprising:
2% of mequinol;
0.10% of adapalene;
5% of ethanol;
from 1% to 2% of at least one gelling agent;
from 0.1% to 0.4% of an antioxidant;
0.10% of EDTA;
from 5% to 15% of an oily liquid phase.

12. The aqueous-alcoholic gel or gel-cream as defined by claim 1, further comprising at least one sulfite salt.

13. The aqueous-alcoholic gel or gel-cream as defined by claim 1, said adapalene being homogenously dispersed therein.

14. A method for formulating the aqueous-alcoholic gel or gel-cream as defined by claim 4, comprising the following successive stages:
a) prepare the formulation phase comprising water and the chelating agent, stir until dissolved, and optionally add a moisturizer and an anti-irritant;
b) heat the mixture from stage (a) to 60° C. and sprinkle in the gelling agent or agents, stirring until homogeneous;
c) leave the mixture to return to room temperature, while stirring;
d) prepare, separately, a first active phase comprising mequinol and alcohol, with stirring until completely dissolved;
e) add this first active phase to the formulation phase after it has returned to room temperature, and maintain stirring;
f) prepare, separately, a second active phase comprising adapalene, surface-active wetting agent and moisturizer, stirring until a smooth, homogeneous dispersion is obtained;
g) then add this second active phase to the formulation phase after it has returned to room temperature, and maintain stirring;
h) neutralize with a neutralizing agent to obtain the desired pH while stirring;
i) add antioxidants to the formulation phase, while stirring.

15. The method as defined by claim 14, wherein an oil phase, obtained by mixing together an oil, a surfactant and a preservative heated to 60° C., is added to the formulation phase obtained at the end of stage (b).

16. A regime or regimen for the treatment of a skin pigmentation disorder, comprising topically applying onto the affected skin area of an individual in need of such treatment, a thus effective amount of the aqueous-alcoholic gel or gel-cream as defined by claim 1.

17. A regime or regimen for the treatment of melasma, chloasma, lentigines, lentigo senile, vitiligo, freckles, post-inflammatory hyperpigmentations due to abrasion, burns, scars, a dermatosis, a contact allergy, naevi, genetically-determined hyperpigmentations, hyperpigmentations of metabolic origin or medication-induced, melanomas or other hyperpigmentation lesions, comprising topically applying onto the affected skin area of an individual in need of such treatment, a thus effective amount of the aqueous-alcoholic gel or gel-cream as defined by claim 1.

18. A regime or regimen for photoprotecting the skin against the damaging effects of exposure to the sun, comprising topically applying thereon a thus effective amount of the aqueous-alcoholic gel or gel-cream as defined by claim 8.

19. A regime or regimen for preventing and/or combating the harmful effects of photoinduced or chronological skin aging, comprising topically applying onto the affected skin area of an individual in need of such treatment, a thus effective amount of the aqueous-alcoholic gel or gel-cream as defined by claim 1.

20. A regime or regimen for cosmetically embellishing the skin and/or enhancing the surface appearance thereof, comprising topically applying onto the skin of an individual in need of such treatment, a thus effective amount of the aqueous-alcoholic gel or gel-cream as defined by claim 1.
